(19) United States
(12) Patent Application Publication
(10) Pub. No.: US 2010/0280459 A1
(43) Pub. Date: Nov. 4, 2010

(54) Medical Tube Support and Method of Use of Same

(76) Inventor: Janet M. Werner, St. Mary's, PA (US)

Correspondence Address: Joseph F. Gula, III, 120 West Tenth Street, Erie, PA, 16501, US

(21) Appl. No.: 12/772,215

(22) Filed: May 2, 2010

Related U.S. Application Data
(60) Provisional application No. 61/175,017, filed on May 3, 2009.

(51) Int. Cl. A61M 39/00; F16L 3/08

(52) U.S. Cl. 604/174; 248/74.2

(57) ABSTRACT

The medical tube support disclosed herein acts as a stabilizer for various types of medical tubing. The invention is designed to attach the tubing to the patient's clothing or bedding to help maintain the integrity of medical access lines and to help alleviate patient discomfort. The invention is also useful for preventing inadvertent pulling of lines at entrance sites, site irritation from gravitational pull and the unintentional entanglement of the tubing. The innovative clip disclosed herein can be used in conjunction with any medical tubing such as gastronomy lines, nasogastric tubing, oxygen lines, various intravenous lines and even suprapublic lines. Patients find the device eases irritation by stabilizing the tubing and eliminates pulling. Medical staff will employ this product to decrease the instances of tangled lines and to reduce the occurrences of misplacement or dislodgment. The design allows for a patient or medical provider to easily wrap the tubular coil mounted at the top of the clip around the medical tubing anywhere near a patient's clothing and to secure the clip portion to said clothing or nearby bedding.

### CROSS-REFERENCE TO RELATED APPLICATIONS

The present application claims the benefit of U.S. Provisional Application No. 61,175,017 filed on May 3, 2009, which is incorporated herein by reference.

### FEDERALLY SPONSORED RESEARCH

Not Applicable

### BRIEF DESCRIPTION OF THE DRAWINGS

These and other features, aspects and advantages of the present invention will become better understood with reference to the following description, appended claims and accompanying drawings where:

FIG. 1 is a side view of the preferred embodiment of the invention.

FIG. 2 is a top view of the preferred embodiment of the invention.

FIG. 3 is a perspective view of the invention without its top mount and coil to allow a view of the interior components of the invention.

FIG. 4 is a perspective view of the invention engaged with medical tubing.

FIG. 5 is a close up view of the coil portion of the invention.

### DESCRIPTION

### Background

There are many different types of medical tubing, all of which serve different purposes. Examples of different lines include gastrostomy, nasogastric, oxygen and intravenous lines. Gastrostomy lines run from a patient's abdomen to a collection bag. Nasogastric lines permit nourishment to run directly into a patient's stomach through the nose. Intravenous lines run from a fluid source into a patient's veins via either a needle or port site. Oxygen tubes import oxygen into the lungs from an external source through a patient's nose.

Each type of tubing presents its own complications and drawbacks. The gravitational pull on tubing such as gastrostomy lines, nasogastric lines and intravenous tubes can cause severe irritation to the patient's skin and may lead to the misplacement or dislodgment of the tubing. Oxygen tubes tend to pull to the side where the oxygen machine is attached, causing soreness and open wounds behind a patient's ear. The constant pressure exerted by medical tubing can lead to more complications for the patient. Tubing also has a tendency to tangle and/or kink, which can make it difficult for the tubing to perform its necessary function. A clipping device that will not puncture the tubing and can easily prevent sliding and abnormal pulling will benefit both medical staff and patients alike.

The Medical Tube Support disclosed herein is a stabilizer for various types of medical tubing. The invention is designed to attach the tubing to the patient's clothing or bedding, to help maintain the integrity of medical access lines and to help alleviate patient discomfort. The clip is also useful for preventing inadvertent pulling of lines at entrance sites, site irritation from gravitational pull and the unintentional entanglement of tubing. Further, a common medical practice is to tape the tubing lines to the patient's skin or clothing or to an external point, all leading to additional problems such as chafing, allergic potential and lack of mobility caused from the excessive taping.

The innovative clip disclosed herein can be used in conjunction with any medical tubing such as gastrostomy lines, nasogastric tubing, oxygen lines, various intravenous lines and suprapubic tubes. Patients will find that this device eases irritation by stabilizing the tubing and eliminating pulling. Medical staff will employ this product to decrease instances of tangled lines and to reduce the occurrences of misplacement or dislodgment.

The design of the device allows for either a patient or a medical provider to easily wrap the tubular coil mounted at the top of the clip around the tubing anywhere near a patient's clothing and to secure the clip portion to said clothing or nearby bedding. For the foregoing reasons, there is a need for an innovative clip that can be produced inexpensively to secure medical tubing to or near a patient's person to allow comfort, safety and mobility when undergoing treatment.

### DETAILED DESCRIPTION OF THE INVENTION

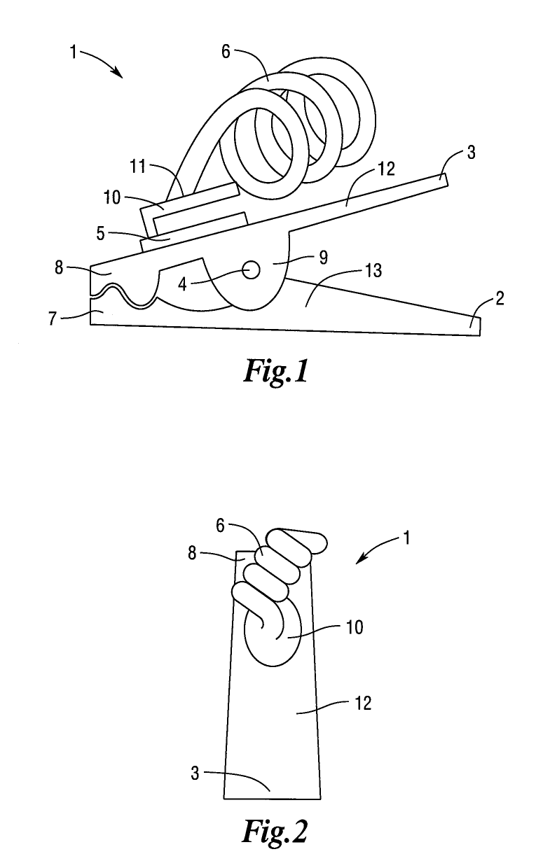

As shown in FIG. 1, a medical tube support apparatus 1 comprises a top member 12 and a bottom member 13 attached at a pivot point 4. There is a spring member behind the pivot point housing 9 which permit said top member 12 to be clamped down at a clamping point 8 with the lower member 13 clamping point 7. On the outer surface of the top member 12 is the circular mount 5 which is permanently attached to said top member 12. In the preferred embodiment of the invention, the circular mount 5 is rotationally attached to a circular component 10. Attached at a seam point 11 is a semi-rigid tubular coil 6. The top member 12 has a distal end 3 and the bottom member 13 has a distal end 2. By applying pressure to the distal end 3 and the distal end 2, the clamping point 8 and clamping point 7 are opened to allow the apparatus to be attached to either clothing or similar material. The coil 6 is then wrapped around a nearby portion of medical tubing to secure said tubing in the proximate vicinity to the patient's clothing.

FIG. 2 shows the apparatus 1 from above whereby the coil 6 is attached to the circular component 10 such that the distal end of said coil 6 extends over the clamping point 8 of the top member 12 rather than the distal end 3. The rotational nature of the circular component 10 allows for said coil 6 to engage with the medical tubing at any angle relative to the support apparatus 1.

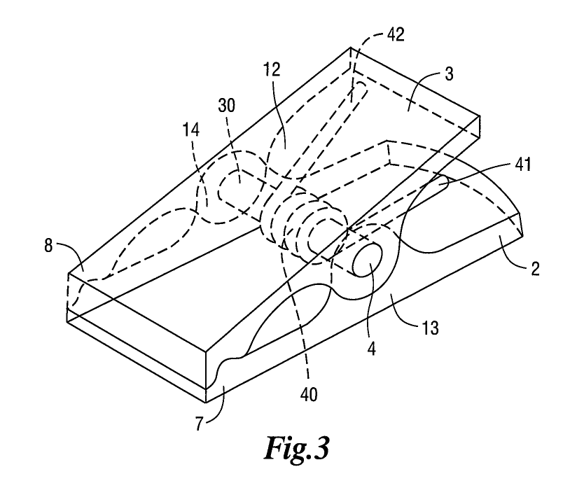

FIG. 3 shows the present invention without the mounts and coil attached to the top member 12 to allow a view of the internal components of the apparatus. Extending internally from the pivot point 4 is a cylinder 30. Wrapped around the cylinder 30 is a spring member 40 with elongated members 41, 42. The elongated members 41, 42 are positioned with respect to the distal end 2 of the bottom member 13 and the distal end 3 of the top member 12 so that the spring member 40 normally urges the clamping points 8, 7 toward one another in a closed position.

The spring member 40 is a spring loaded pivot pin that is conventionally made from metal and includes a body portion that surrounds a cylinder 30. As in FIG. 3, the spring member 40 is in the relaxed position and wound to the operative position with the elongated member 41 being roughly parallel and pointing in the same direction as elongated member 42 to provide the conventionally understood spring effect.

In order to clip the apparatus to any base material, such as a patient's clothing or nearby bedding materials, pressure is applied to the distal end 3 of the top member 12 and the distal end 2 of the bottom member 13 which opens the clamping points 7, 8 to allow the apparatus to engage with the base materials. Applying pressure to said distal ends 2, 3 causes the elongated members 41, 42 of the spring member 40 to be compressed such as to urge the clamping points 7, 8 away from one another into an open position. Once in an open position, the space created between the clamping points 7, 8 allows for a base material such as bedding or clothing to be so inserted. Once the base material is inserted between the open clamping points 7, 8, the pressure is removed from the distal ends 2, 3 of the bottom member 13 and top member 12 so that the natural pressure exerted by the elongated members 41, 42 of the spring member 40 urges the clamping points 7, 8 into their closed position around the base materials.

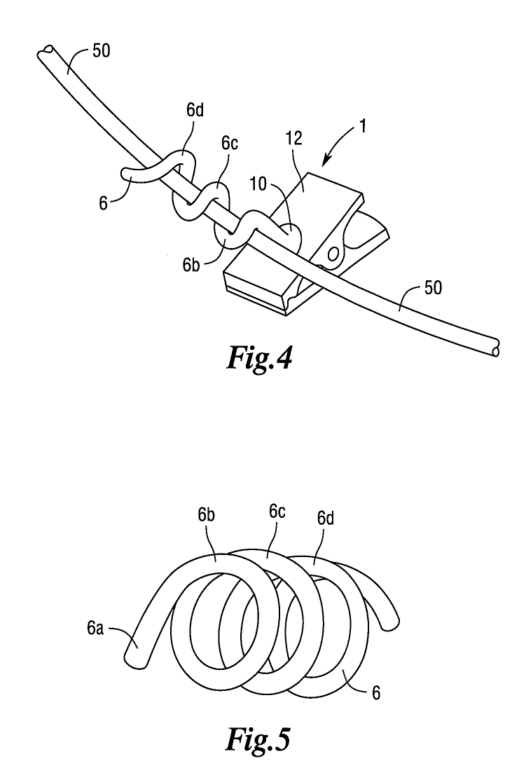

The coil 6 is made of a semi-rigid material that holds its coil form but can be partially unraveled to permit said coil to be wrapped around a tubular structure. FIG. 4 shows the apparatus 1 engaged with medical tubing 50. The top member 12 is shown with the circular component 10 attached to the coil 6. The coil 6 has been partially unraveled so it is engaged with the tubing 50 to hold the tubing 50 in place. To engage the coil 6 with the tubing 50, the coil is partially uncoiled and pressure is placed first on the first area of curvature 6b, then on the area of curvature 6c and then on the area of curvature 6d such that the coil 6 is engaged around the circumference of the tubing 50.

The tubular coil can be attached to the circular component 10 or circular mount 5 by either fusing the proximal end of the coil 6a by heat, welding, glue bonding or other adhesive means. As shown in FIG. 5, the adhesive means causes the coil 6 to be permanently joined at the proximal end 6a to the circular component 10 or circular mount 5.

The elongated members 12, 13 can be manufactured from a plastic-like polymer or similar material to provide the necessary stability for use as a clip in the present invention. The spring member 40 and the cylinder 30 can be manufactured from metal sufficiently strong to allow the conventionally understood spring effect. The spring effect described herein can be accomplished by other means including a spring-forming web known by others skilled in the art.

In another embodiment of the present invention, the apparatus 1 can be manufactured with a highly visible color to indicate to caregivers and patients that the apparatus 1 is attached to the medical tubing 50 and needs to be addressed when the tubing is removed, changed or altered in any way. The coloring of the apparatus 1 can be achieved either utilizing a colored plastics polymer or similar material, or by coating the apparatus 1 with a permanent paint or similar spray.

What is claimed is:

1. An apparatus for supporting medical tubing to alleviate discomfort and maintain tubing integrity, the apparatus comprising:
a clip for releasably attaching the apparatus to the patient's clothing or bedding materials, wherein the clip comprises a pair of elongated members pivotally attached, wherein each elongated member has an outer surface, an inner surface, two side surfaces, a clamping end and a distal end;
a spring for applying and releasing pressure on the distal ends of said elongated members;
a circular mount having a top surface and a bottom surface permanently mounted to the outer surface of the top elongated member; and
a tubular coil attached to said circular mount's top surface to engage the medical tubing.

2. The apparatus of claim 1 wherein said the top surface of the circular mount is rotationally mounted to a circular component to allow said circular component to rotate 360°.

3. The apparatus of claim 1 wherein said elongated members, said circular member and said tubular coil are manufactured from materials which are of a highly visible color.

4. The apparatus of claim 2 wherein said elongated members, said circular member and said tubular coil are manufactured from materials which are of a highly visible color.

5. The apparatus of claim 1 wherein said elongated members, said circular member and said tubular coil are coated with a highly visible color.

6. The apparatus of claim 2 wherein said elongated members, said circular member and said tubular coil are coated with a highly visible color.

7. The apparatus of claim 1 wherein said spring is a spring forming web.

8. The apparatus of claim 2 wherein said spring is a spring forming web.

9. A method of supporting medical tubing on a patient to alleviate discomfort and maintain tube integrity, the method comprising:
providing an apparatus having a clip comprising a pair of elongated members pivotally attached wherein each elongated member has an outer surface, an inner surface, two side surfaces, a clamping end and a distal end, a spring, a circular mount having a top surface and a bottom surface permanently mounted to the outer surface of the top elongated member and a tubular coil attached to said circular mount's top surface;
applying pressure simultaneously to the distal ends of said elongated members to allow the clamping ends to move away from each other,
sliding the apparatus onto a patient's clothing securely;
releasing pressure from said distal ends of said elongated members;
applying pressure on the first area of curvature of said tubular coil to allow said coil to wrap around the circumference of medical tubing; and
applying pressure on the successive areas of curvature of said tubular coil to allow said coil to wrap around the circumference of medical tubing.

10. The method of claim 9 wherein the improvement is providing said coil attached rotationally to said circular mount.
